(19) United States
(12) Patent Application Publication
(10) Pub. No.: US 2001/0025192 A1
(43) Pub. Date: Sep. 27, 2001

(54) Single and multi-polar implantable lead for sacral nerve electrical stimulation

(75) Inventors: Martin T. Gerber, Maple Grove, MN (US); John M. Swoyer, Andover, MN (US)

Correspondence Address: Eric R. Waldkoetter, Medtronic, Inc., 710 Medtronic Parkway, Minneapolis, MN, 55432-5604, US

(73) Assignee: Medtronic, Inc.

(21) Appl. No.: 09/843,051

(22) Filed: Apr. 26, 2001

Related U.S. Application Data
(63) Continuation-in-part of application No. 09/531,041, filed on Mar. 20, 2000.
Application No. 09/531,041 is a division of application No. 09/301,937, filed on Apr. 29, 1999, now Pat. No. 6,055,456.

(51) Int. Cl. A61N 1/05

(52) U.S. Cl. 607/117

(57) ABSTRACT

An implantable medical lead for stimulation of the sacral nerves comprises a lead body which includes a distal end and a proximal end, and the distal end having at least one electrode contact extending longitudinally from the distal end toward the proximal end. The lead body at its proximal end may be coupled to a pulse generator, additional intermediate wiring, or other stimulation device. The electrode contact of the permanently implantable neurostimulation lead comprises an elongated, flexible, coiled wire or mesh electrode having an exposed electrode length that is adapted to be inserted through the foramen from a posterior access to locate the coiled wire electrode alongside the sacral nerve extending anteriorly and/or posteriorly therefrom. The coiled wire or mesh electrode structure is flexible and bendable to enable its placement through through the foramen and alongside the sacral nerve and to conform to the surrounding nerves and tissue. Preferably, further shorter length electrodes are provided along the distal segment of the lead body to enable testing of the positioning of the elongated wire coil or mesh electrode or to provide alternate stimulation electrodes upon dislocation of the elongated wire coil or mesh electrode.

This is a continuation-in-part of co-pending U.S. patent application Ser. No. 09/531,041 filed Mar. 30, 2000, which is a division of U.S. patent application Ser. No. 09/301,937 filed Apr. 29, 1999, now U.S. Pat. No. 6,055,456.

### BACKGROUND OF THE INVENTION

1. Field of the Invention

This invention relates generally to an apparatus that allows for stimulation of the sacral nerves. More specifically, this invention relates to an implantable medical lead having at least one stimulation electrode wherein the lead is implanted near the sacral nerves for stimulation of a bundle of sacral nerve fibers. Moreover, this invention relates to the method of implantation and anchoring of the medical lead near the sacral nerve to allow for stimulation.

2. Description of Related Art

Pelvic floor disorders such as, urinary incontinence, urinary urge/frequency, urinary retention, pelvic pain, bowel dysfunction (constipation, diarrhea), erectile dysfunction, are bodily functions influenced by the sacral nerves. Specifically, urinary incontinence is the involuntary control over the bladder that is exhibited in various patients. Incontinence is primarily treated through pharmaceuticals and surgery. Many of the pharmaceuticals do not adequately resolve the issue and can cause unwanted side effects, and a number of the surgical procedures have a low success rate and are not reversible. Several other methods have been used to control bladder incontinence, for example, vesicostomy or an artificial sphincter implanted around the urethea. These solutions have drawbacks well known to those skilled in the art. In addition, some disease states do not have adequate medical treatments.

In one current method of treatment for incontinence using electrical stimulation, two stimulation systems are implanted each having an implantable lead with discrete electrodes positioned directly on selected sacral nerves for sphincter and bladder stimulation respectively. Typically, the electrodes at the distal ends of the leads are formed as bands that encircle the nerves. The leads are connected to a pulse generator wherein an electrical stimulation pulse is transmitted. The sphincter is stimulated to prevent incontinence by application of electrical stimulation pulses to the sphincter function controlling electrode. When it is desired to evacuate the bladder, the electrical pulse to the sphincter function controlling electrode is halted, and electrical stimulation pulses are delivered to the bladder function controlling electrode. After a delay, the bladder stimulation is discontinued and the sphincter is again stimulated.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Thus, for example, medical leads having discrete electrode contacts have been implanted on and near the sacral nerves of the human body to provide partial control for bladder incontinence. Unlike other surgical procedures, sacral nerve stimulation using an implantable pulse generator is reversible by merely turning off the pulse generator. Several techniques of electrical stimulation may be used, including stimulation of nerve bundles within the sacrum. The sacrum, generally speaking, is a large, triangular bone situated at the lower part of the vertebral column, and at the upper and back part of the pelvic cavity. The spinal canal runs throughout the greater part of the sacrum. The sacrum is perforated by the anterior and posterior sacral foramina that the sacral nerves pass through.

Several systems of stimulating sacral nerves have been disclosed. For example, U.S. Pat. Nos. 4,771,779 and 4,607,739 to Tanagho et al. and the related U.S. Pat. No. 4,739,764 to Lue et al., all incorporated herein by reference, disclose implanting an electrode on at least one nerve controlling the bladder. In one embodiment, a lead bearing a distal stimulation electrode is percutaneously implanted through the dorsum and the sacral foramen of the sacral segment S3 for purposes of selectively stimulating the S3 sacral nerve. The single distal tip electrode is positioned using a hollow spinal needle through a foramen (a singular foramina) in the sacrum. The electrode is secured by suturing the lead body in place However, the lead depicted in FIG. 5 of the 779 patent appears to have a single discrete tip electrode that would be sensitive to movement and dislodgement from the most efficacious location due to stresses placed on the lead by the ambulatory patient despite the suture fixation. Electrodes positioned within the sacrum to control bladder function are also disclosed in U.S. Pat. No. 4,569,351 to Tang, incorporated herein by reference.

The current lead designs used for sacral nerve stimulation through a foramen uses four ring-shaped, stimulation electrodes spaced along a distal segment of the lead body to provide a distal electrode array less sensitive to electrode movement. During implantation, the physician steers the implantable pulse generator outputs to the electrodes to provide the most efficacious therapy, and the selection of the electrodes can be changed if efficacy using a selected electrode fades over time.

In one version, each electrode is 0.118 inches (3.0 mm) long, and the electrodes are spaced apart by 0.118 inches (3.0 mm) along the distal electrode segment of the lead body. In another version, each electrode is 0.236 inches (6.0 mm) long, and the electrodes are spaced apart by 0.236 inches (6.0 mm) along the distal segment of the lead body. Each distal electrode is electrically coupled to the distal end of a lead conductor within the elongated lead body that extends proximally through the lead body. The proximal ends of the separately insulated lead conductors are each coupled to a ringshaped connector element in a proximal connector element array along a proximal segment of the lead body that is adapted to be coupled with the implantable neurostimulation pulse generator or neurostimulator.

Electrical stimulation pulses generated by the neurostimulator are applied to the sacral nerve through one or more of the distal electrodes in either a unipolar or bipolar stimulation mode. In one unipolar stimulation mode, the stimulation pulses are delivered between a selected active one of the distal electrodes and the electrically conductive, exposed surface of the neurostimulator pulse generator housing or can providing a remote, indifferent or return electrode. In this case, efficacy of stimulation between each distal electrode and the neurostimulator pulse generator can electrode is tested, and the most efficacious combination is selected for use. In a further unipolar stimulation mode, two or more of the distal electrodes are electrically coupled together providing stimulation between the coupled together distal electrodes and the return electrode. In a bipolar stimulation mode, one of the distal lead electrodes is selected as the indifferent or return electrode. Localized electrical stimulation of the sacral nerve is effected between the active lead electrode(s) and the indifferent lead electrode. Again, testing of stimulation efficacy is undertaken to ascertain the most efficacious combination of lead electrodes.

A problem associated with the prior art electrical stimulation to control incontinence is positioning and maintaining the discrete ring-shaped lead electrode(s) in casual contact, that is in location where slight contact of the electrode with the sacral nerve may occur or in close proximity to the sacral nerve to provide adequate stimulation of the sacral nerves. Another problem is providing constant or consistent stimulation while allowing some movement of the lead body.

The current electrical designs used for sacral nerve stimulation are not optimized for the application because the small size of the electrode(s) make them sensitive to minor motions of the electrode(s) and or lead relative to the target nerve.

Additionally, physicians spend a great deal of time with the patient under a general anesthetic placing the leads due to the necessity of making an incision exposing the foramen and due to the difficulty in optimally positioning the small size stimulation electrodes relative to the sacral nerve. The patient is thereby exposed to the additional dangers associated with extended periods of time under a general anesthetic. Movement of the lead, whether over time from suture release or during implantation during suture sleeve installation, is to be avoided. As can be appreciated, unintended movement of any object positioned proximate a nerve may cause unintended nerve damage. Moreover reliable stimulation of a nerve requires consistent nerve response to the electrical stimulation that, in turn, requires consistent presence of the electrode portion of the lead proximate the sacral nerve. But, too close or tight a contact of the electrode with the sacral nerve can also cause inflammation or injury to the nerve diminishing efficacy and possibly causing patient discomfort.

Accordingly, there remains a need in the art for an implantable electrical lead that allows for stimulation of a bundle of nerves and allows for some movement after implantation and is capable of accommodating to the sacral nerve to avoid injury or discomfort.

### SUMMARY OF THE INVENTION

The present invention recognizes and provides a solution to the problems associated with implanting and maintaining electrical leads in close proximity or casual contact with discrete nerve fibers of the sacral nerves by providing a unique solution that allows implantation near to, but avoiding compressive contact with, the sacral nerves. Additionally, the invention provides a method of implanting a medical electrical stimulation lead through the foramen for control of incontinence by stimulating a bundle of nerve fibers of the sacral nerve anterior to the sacral nerve opening through the sacrum.

Briefly, one embodiment of the present invention comprises a permanently implantable neurostimulation lead with at least one elongated, flexible, coiled wire electrode having an exposed coil length that is adapted to be inserted through the foramen from a posterior access to locate the coiled wire electrode alongside the sacral nerve extending anteriorly and or posteriorly therefrom. The coiled wire electrode structure is flexible and bendable to enable its placement through the foramen and alongside the sacral nerve and to conform to the surrounding nerves and tissue

Preferably, the neurostimulation lead of this embodiment of the present invention is formed having an elongated stimulation electrode formed of a flexible wire conductor wound about or inserted a distal segment of the lead body to form an exposed electrode having a coiled wire electrode length. At least one end of the coiled wire electrode is electrically and mechanically connected at an annular connection zone with a band or ring-shaped electrode connector that may be exposed to further extend the electrode surface area or may be insulated. The electrode connector is in turn connected to the distal end of a lead conductor extending proximally through the lead body to a connector element at a proximal connector segment of the lead.

In a further embodiment of the present invention, a permanently implantable neurostimulation lead is provided with at least one elongated distal mesh electrode in a distal segment of the lead body. A lead conductor extends between a proximal connector element and the distal mesh electrode. The distal mesh electrode further preferably comprises an elongated tube surrounding the lead body and electrically connected to the lead conductor. The elongated tube has a sidewall formed of a lattice framing windows extending through the sidewall and imparting flexibility to the elongated distal mesh electrode.

The neurostimulation lead of the present invention can be implemented having a single elongated mesh or coiled wire stimulation electrode as described or with a plurality of such elongated coiled wire conductors spaced apart along the distal electrode segment of the lead body. Preferably, the neurostimulation lead of the present invention can be implemented having a single elongated mesh or coiled wire stimulation electrode as described along with a plurality of ring-shaped distal electrodes spaced apart from one another in the distal electrode region. This allows the advantages of the extended, flexible electrode length while providing an option for bipolar stimulation or redundant back-up electrodes along an appropriate length.

Each distal electrode is electrically coupled to the distal end of a lead conductor within the elongated lead body that extends proximally through the lead body. The proximal ends of the separately insulated lead conductors are each coupled to a ringshaped connector element in a proximal connector element array along a proximal segment of the lead body that is adapted to be coupled with the implantable neurostimulation pulse generator or neurostimulator. Electrical stimulation pulses generated by the neurostimulator are applied to the sacral nerve through one or more of the distal electrodes in either a unipolar or bipolar stimulation mode.

The flexible elongated mesh or wire coil electrodes can bend somewhat to fit through a foramen to locate the elongated electrode optimally with respect to a sacral nerve. Accordingly, the present invention advantageously provides a unique implantable medical electrical stimulation lead that provides adequate stimulation of the sacral nerves for control of incontinence and other pelvic floor disorders with the sacral nerves and with less sensitivity to placement. The unique lead simplifies the implant procedure and reduces or eliminates the need to reprogram the implantable pulse generator stimulation levels or re-open the patient to move the lead.

The implantation method for implanting the lead of the present invention allows more rapid placement of the electrodes for the treatment of incontinence whereby the lead is placed near the sacral nerves. Implanting the medical electrical lead near the sacral nerves with less specificity as to location near the sacral nerves reduces the time for implantation. Currently, the implantation procedure for existing medical electrical leads stimulating the sacral nerve fibers takes approximately 20-60 minutes. The present invention allows for implantation near the sacral nerve bundle and reduces the time for implantation to approximately 5-10 minutes. The elongated electrode surface area of the coiled wire electrode creates a wider electric field which allows the lead to be placed in a less precise or gross manner while still providing adequate electrical stimulation to the sacral nerve.

Yet another object of this invention is to provide a medical electrical lead and method of implantation whereby the lead can allow for some movement of the lead without deteriorating the capture of the sacral nerves. Because the electrode does not need to be in direct contact with the nerve fibers and due to the large electrode area, a small amount of movement from the original implant position does not reduce the nerve capture.

A further object of this invention is to provide a medical electrical lead for stimulating the sacral nerves having a smaller than typical diameter. Providing the medical electrical lead with a smaller diameter may allow for alternate less invasive implantation techniques such as the use of a cannula. The smaller diameter medical electrical lead provides less trauma to a patient during implantation. Using this system for implantation may allow the physician to use a local anesthesia instead of a general anesthesia thus reducing the dangers inherent with the use of a general anesthetic. The full range of advantages, and features of this invention are only appreciated by a full reading of this specification and a full understanding of the invention. Therefore, to complete this specification, a detailed description of the invention and the preferred embodiments follow, after a brief description of the drawings, wherein additional advantages and features of the invention are disclosed.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

Preferred embodiments of the invention are illustrated in the drawings, wherein like reference numerals refer to like elements in the various views, and wherein:

FIG. 1 is a plan view of the lead with one electrode contact extending from the distal end.

FIG. 2 is a plan view of the lead with one electrode extending from the distal end and including an anchoring mechanism.

FIG. 3 is a plan view of the lead having two electrode contacts to provide for a bipolar configuration.

FIG. 4 is a plan view of the lead adapted to accept a stylet.

FIG. 5 is a plan view of the lead adapted to accept a stylet and having a curved distal end.

FIG. 6 is a schematic illustration of a lead implanted near the sacral nerve.

FIG. 7 is a plan view of one embodiment of a neurostimulation lead of the present invention having a coiled wire electrode and a plurality of ring electrodes.

FIG. 8 is a cross-section view of the construction of the lead body and proximal ring electrodes taken along lines 8-8 of FIG. 7.

FIG. 9 is a cross-section view of the construction of the lead body and distal wire coil and ring electrodes taken along lines 9-9 of FIG. 7.

FIG. 10 is a side view of the wire coil electrode attached to a ring-shaped electrode connector for connection with an internally disposed lead conductor.

FIG. 11 is an end view of the ring-shaped electrode connector of FIG. 10.

FIG. 12 is an enlarged detail view of the connection of the distal end of the lead conductor with the ring-shaped electrode connector of FIGS. 10 and 11.

FIG. 13 is an enlarged perspective view of an alternative form of the flexible elongated electrode comprising an elongated wire mesh electrode that may be substituted for the wire coil electrode.

### DESCRIPTION OF THE PREFERRED EMBODIMENTS

Referring to FIG. 1, an implantable medical lead 10 that allows for non-direct contact stimulation of the sacral nerves comprises a lead body 15 having at least one electrode contact or electrode 20 and a distal end 25. The electrode contact 20 extends longitudinally for a length of between 0.10 inches and 1.50 inches from the distal end 25 toward a proximal end 35. The distal end 25 of the lead body 15 may comprise an electrically conductive or non-conductive tip 30.

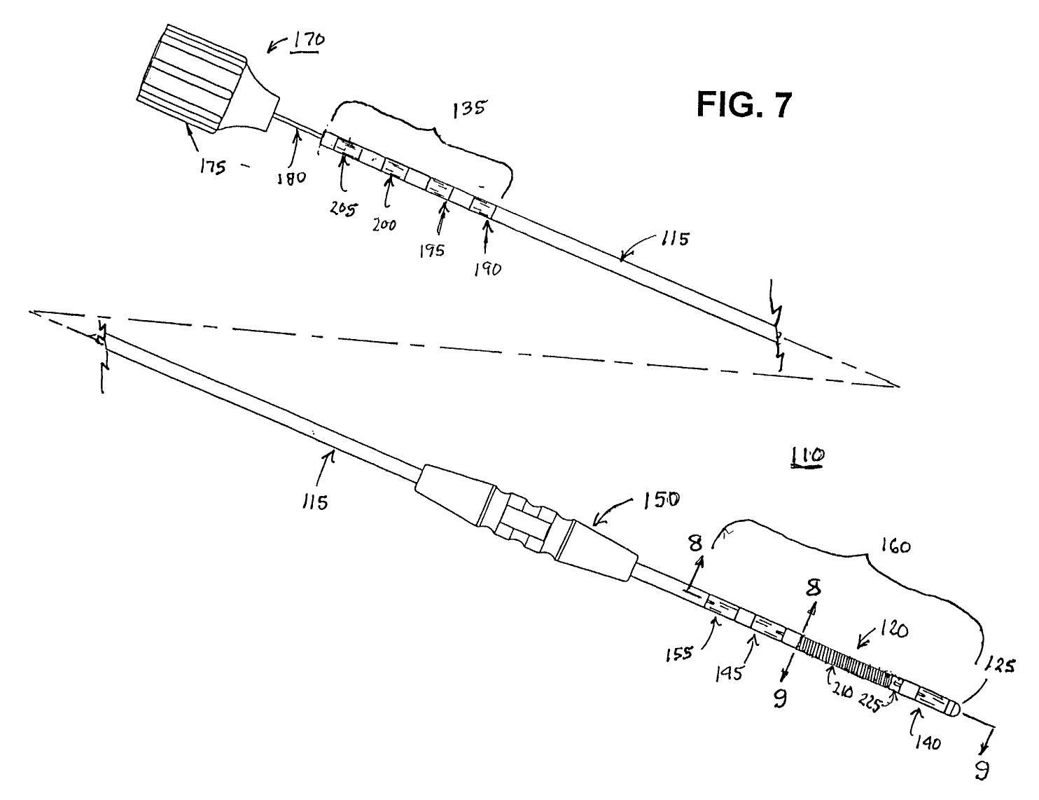

The proximal end 35 of the lead body 15 bears proximal connector elements (not shown) of the type described below with respect to FIG. 7 that may be coupled to a neurostimulation pulse generator, additional intermediate wiring, or other stimulation device. An example of such an implantable pulse generator is the Medtronic InterStim Neurostimulator Model 3023. The stimulation pulses produced by the pulse generator coupled to the connector element at the proximal end 35 of the lead body 15 are conducted through a lead conductor in the lead body 15 to the electrode 20. One preferred embodiment of the electrode 20 is 0.40 inches long. The current typical lead for stimulation of the sacral nerves includes a discrete electrode. The larger electrode contact 20 of this invention generates a larger electric field for stimulating the sacral nerve. The larger electric field makes it easier to stimulate the nerve bundle. The implantation process is simplified because this medical lead 10 does not require the specificity of location of the small sized electrodes of current leads.

In one preferred embodiment, the elongated electrode contact 20 is made of a solid surface, bio-compatible material, e.g., a tube formed of platinum, platinum-iridium, and stainless steel that does not degrade when electrical stimulation is delivered through it. Preferably the elongated electrode contact or electrode 20 is made up of a flexible structure, e.g., a coiled wire or a wire mesh, formed of the same or similar bio-compatible materials.

The lead body 15 of the present invention comprises one or more conductor wire(s) within an insulating sheath. The conductor material is preferably an MP35N alloy. The lead body 15 insulation material is preferably polyurethane or silicone. Other suitable materials known to those in the art may also be used. A typical diameter of the lead body 15 is 0.050 inches but a smaller diameter is also acceptable.

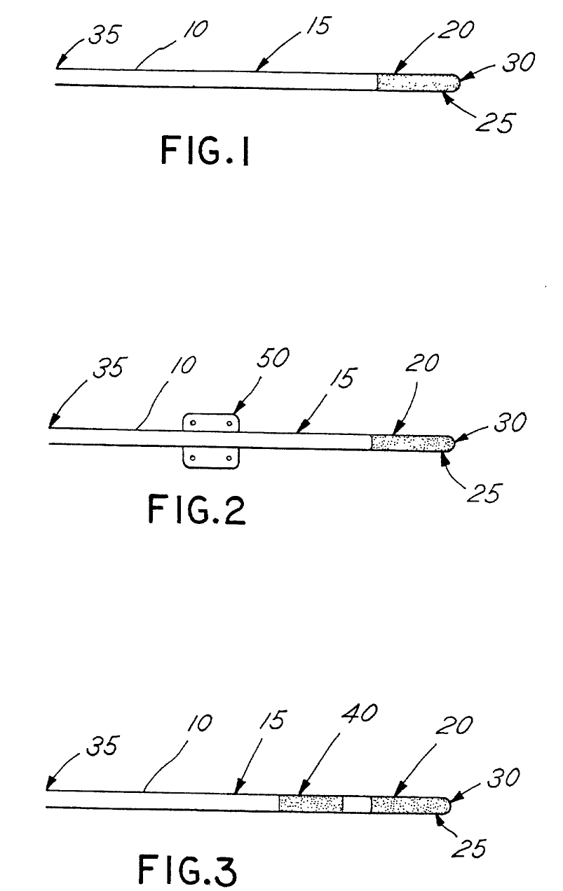

Referring to FIG. 2, the implantable medical lead 10 of the present invention may have an anchoring mechanism 50 to fixate the medical lead 10 in the desired position. The anchoring mechanism 50 is a molded part, integral to the medical lead 10, where the physician can pass the sutures through the molded part to attach the medical lead 10 to the human anatomy. The anchoring mechanism 50 has at least one through hole, shown in FIG. 2, that allows the medical lead 10 to be inserted through the anchoring mechanism before adhering to the body. Another anchoring mechanism 50 is adapted to allow the use of a bone screw to screw to adhere the lead to the sacrum. Another anchoring mechanism 50 includes attaching an anchor to the medical lead 10 during the implantation procedure to allow the physician to suture to the anatomy. Yet another anchoring mechanism 50 is to allow the medical lead 10 to fibrose in naturally using the human body's natural reaction to a foreign body or healing. A further anchoring mechanism 50 is to use enzyme glues to provide the necessary anchoring.

Turning to FIG. 3, the medical lead 10 of the present invention may have two electrode contacts or electrodes 20 and 40. As above, the elongated first electrode contact 20 is preferably 0.40 inches in length. The second ring shaped electrode contact 40 is preferably 0.10 inches in length. The length of the first and the second electrode contacts 20 and 40 extend longitudinally from the distal end 25 toward the proximal end 35. The first electrode contact 20, as above in the single electrode embodiment, begins at the distal end having either a conductive or a non-conductive tip 30. The second electrode contact 40 extends for a length starting at approximately 1.00 inch from the distal end 30 toward the proximal end 35. The first electrode contact and the second electrode contact do not overlap. The second electrode contact extends from a point beyond the end of the first electrode contact toward the proximal end. The length of the second electrode contact 40 is preferably 0.10 inches but may range between 0.03 and 1.00 inches. The length of the second electrode contact 40 must be large enough that the current density is not at a level that causes damage to the tissue or that may be sensed by the patient.

As above, the first and second electrode contacts 20 and 40 can be made of a solid surface material, for example platinum, platinum-iridium, or stainless steel. The first and second electrode contacts 20 and 40 may also be constructed of a coiled wire or wire mesh. Another alternative embodiment of the medical lead 10 includes the first electrode contact 20 comprising a solid surface material and the second electrode contact 40 comprising a coiled wire or wire mesh. A coiled first electrode contact 20 may be preferred from a physiological standpoint whereas a solid second electrode may be preferred from a manufacturing perspective. The preferred embodiment will have a coiled first electrode contact 20 and a solid surface material second electrode contact 40. Where two electrodes are used, the first electrode contact 20 will be one polarity and the can of the implantable pulse generator will be the other polarity. In some instances, where the patient has pain at the implantable pulse generator site caused or increased by the stimulation, the second electrode contact 40 would be used instead of the can of the implantable pulse generator, thus eliminating the pain at the implantable pulse generator site. The first and second electrode contacts 20 and 40 are sized such the first electrode contact 20 does not longitudinally overlap with the second electrode contact 40.

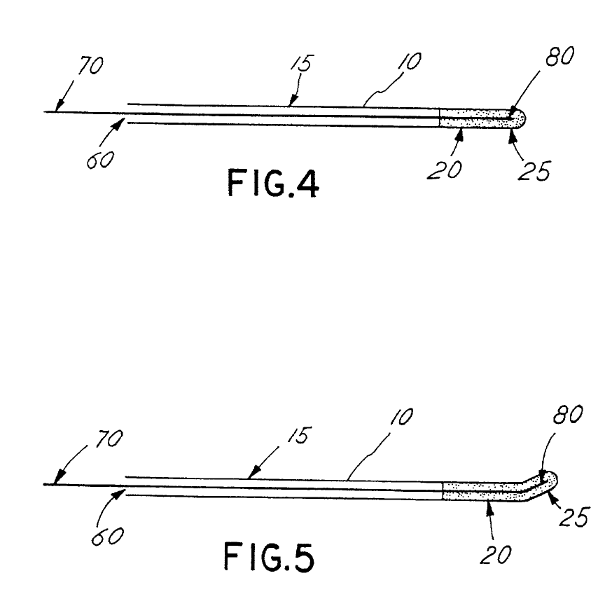

In FIG. 4, the implantable medical lead 10 may include an internal lumen or cavity 60 shaped to accept a stylet 70. The stylet 70 is inserted into the lead body internal cavity 60 prior to implantation. The stylet 70 is made of solid wire such as tungsten or stainless steel. By inserting a stylet 70 into the lead body internal cavity 60, the medical lead 10 is stiffened to provide support to the lead body 15 during implantation. Use of a medical lead 10 with a stylet 70 is particularly useful for implantation using a cannula. The cannula is inserted to extend from the skin to the foramen and enables passage of the lead 10 stiffened by the stylet 70 through the cannula lumen to locate the distal electrode contacts in proximity to or in casual contact with the sacral nerve.

Turning to FIG. 5, the stylet 70 can alternatively have a pre-formed shape. Various shapes of the stylet distal end 80 could be used to assist or guide the placement of the medical lead 10 to the optimal physiological position. An alternative shape of the stylet 70 includes a curved distal end 80. The medical lead 10 may also be manufactured with a pre-bent optimized shape to accept the stylet 70. With a pre-bent medical lead 10, a stylet 70 may or may not be used to assist in the implantation of the lead. A stylet 70 with a straight distal end 80 may be used to straighten the lead for passing through the cannula. The construction of the lead must be adapted to accommodate the stylet 70 to ensure that the stylet 70 does not rupture the insulation on the electrical conductors.

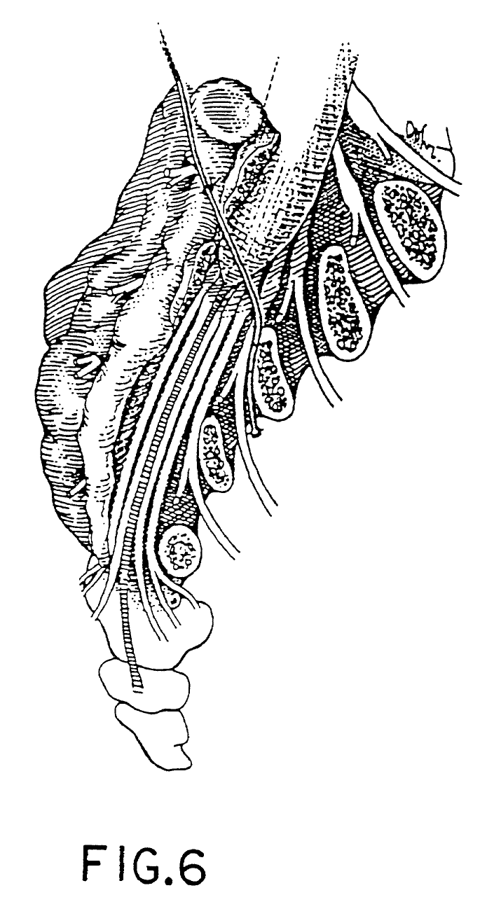

FIG. 6 shows an overall schematic of the sacral nerve area with a medical lead 10 implanted near a sacral nerve for stimulation. The implantable medical lead 10 is inserted by first making an incision appropriate to the size of the patient and then splitting the paraspinal muscle fibers to expose the sacral foramen. The physician then locates the desired position and inserts the medical lead 10 into the foramen and anchors the medical lead 10 in place. The medical lead 10 should be placed close enough to the nerve bundle that the electrical stimulation results in the desired physiological responses. The desired effect varies depending on which pelvic floor disorder is being treated or which nerve is being stimulated. The preferred position for the medical lead 10 is implantation in close proximity of the nerve. This placement of the medical lead 10 to the nerve results in the most efficient transfer of electrical energy. With the medical lead 10 of this invention, the positioning is much less critical than current lead designs.

To determine the best location of the lead, an insulated needle with both ends exposed for electrical stimulation is used to locate the foramen and locate the proximity of the nerve by electrically stimulating the needle using an external pulse generator. The location is tested by evaluating the physiologic response and by the electrical threshold required to get that response. Once the appropriate location has been determined using the insulated needle, the medical lead 10 is implanted in that approximate location. For control of incontinence, the physician preferably implants the medical lead 10 near the S3 sacral nerves. The implantable medical lead 10 may, however, be inserted near any of the sacral nerves including the S1, S2, S3, or S4, sacral nerves depending on the necessary or desired physiologic response. This invention can be used to stimulate multiple nerves or multiple sides of a single nerve bundle. In addition, the medical lead 10 can also be used as an intramuscular lead. This may be useful in muscle stimulation such as dynamic graciloplasty. Placement of the medical lead 10 of this invention does not require the specificity of current electrical stimulation of the sacral nerves. Additionally, the larger electrode contacts 20 and 40 make the present invention less susceptible to migration of the implantable medical lead 10 after implantation.

FIG. 7 depicts a further preferred embodiment of a neurostimulation lead 110 in accordance with the present invention. The illustrated lead 110 comprises an elongated lead body 115 bearing a plurality, e.g., four, distal electrodes 140, 120, 145, and 155 arrayed along distal electrode array segment 160 that are coupled through separately insulated conductors extending to respective proximal connector elements 190, 195, 200 and 205 arrayed along proximal connector element array segment 135. A tubular attachment mechanism 150 is formed around or fitted over a section of the lead body 150. A stylet 170 comprises an elongated stylet wire 180 that can be inserted through or retracted from an axial lumen of the lead body 115 by manipulation of a stylet handle 175 attached at the proximal end of the stylet wire 180.

A wire coil electrode 120 comprises the end-to-end assembly of an elongated flexible wire coil 210 and a more rigid, relatively short, ring or band-shaped, electrode connector 225. The remaining ring electrodes 140, 145, 155 are relatively short and ring or band-shaped. It will be understood that the number, selection and positioning of the ring electrodes and the number and positioning of the coil electrode(s) can be selected to fit the distal electrode array segment 160, and that each such electrode can be fabricated accordance with the following description of the fabrication and construction of the illustrated embodiment.

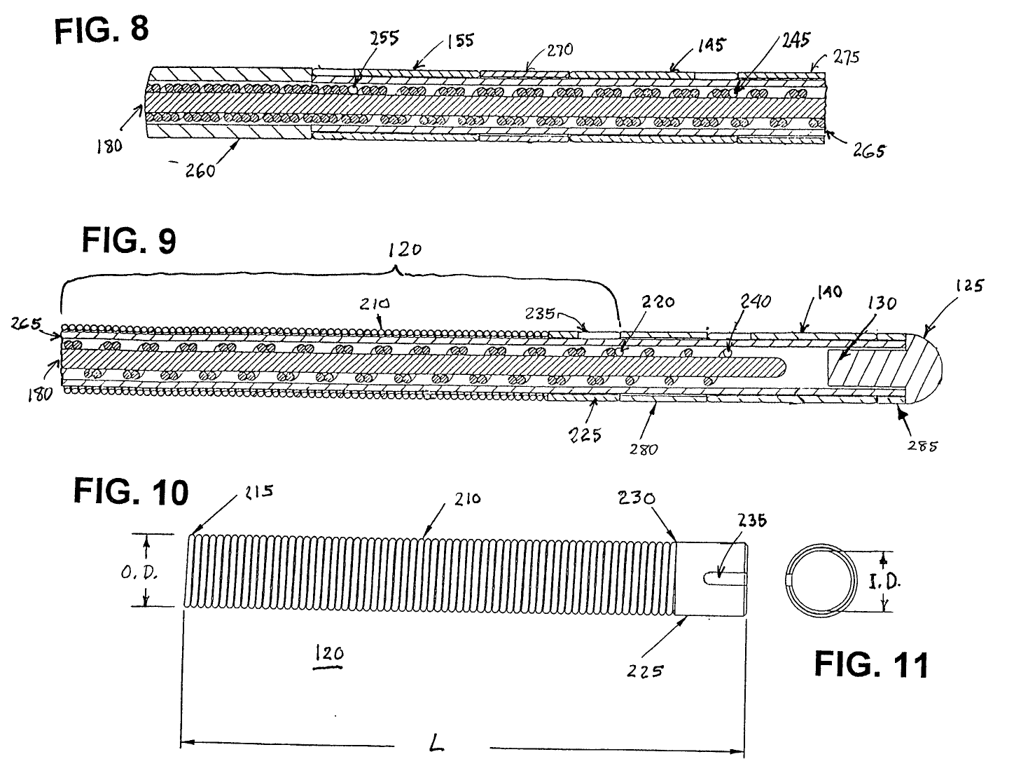

FIGS. 8 and 9 illustrate the lead body fabrication proximal to and within the distal electrode array segment 160. The lead body 115 is formed of a non-conductive, body compatible, flexible, outer tubular sheath 260 extending between the proximal connector element array segment 135 and the distal electrode array segment 160. The outer sheath 260 is preferably formed of polyurethane. The lead body 115 also comprises a non-conductive body compatible, flexible, inner tubular sheath 265 extending from the distal end of the outer tubular sheath 260 through the distal electrode array segment 160 to the tip 130 at the distal lead end 125. The inner tubular sheath 265 supports the electrodes 140, 120, 145 and 155 and a like number of insulator bands 270, 275, 280 and 285 in linear and axial alignment. The proximal portion of the distal tip 130 is inserted into the distal end opening of the inner tubular sheath 265. The inner sheath 265 is preferably formed of polyurethane. A plurality of insulator bands and an inner sheath are also employed in the proximal connector array segment 135 to electrically isolate and support the connector elements 190, 195, 200 and 205 in linear and axial alignment.

A continuous lead lumen is formed by the aligned outer sheath 260 and the inner sheathes that extends from the lead proximal end to the lead distal end 125. The lead conductors 240, 220, 245, and 255 extend through the lumen. The lead conductors 240, 220, 245, and 255 are separately insulated by an insulation coating and are wound in a quadra-filar manner having a common winding diameter. The coil formed by the coiled wire conductors defines the stylet wire lumen of the lead body 115. It will be understood that a further inner tubular sheath could be interposed within the aligned wire coils to provide a stylet lumen.

The elongated wire coil electrode 120 comprises the wire coil 210 and a band or ring-shaped electrode connector 225. The wire coil 210 is formed of a flexible metallic sheath or platinum or platinum alloy wire having a diameter of about 0.1 mm. The wire is wound over a mandrel to form the wire coil 210 having a coil O.D, coil I.D. and a coil length as shown in FIGS. 10 and 11.

One end of the wire coil 210 is electrically and mechanically connected at an annular connection zone 230 with the band or ring-shaped electrode connector 225 having a common I.D. and O.D. with the wire coil 210. The electrical and mechanical connection at the connection zone 230 can be effected by axially aligning and butt-welding and/or adhering the facing ends of the wire coil 210 and the connector 225 together. The electrode connector 225 may be exposed to provide part of the electrode surface area and electrode length L (as shown in FIG. 10) or may be electrically insulated. The outer diameter O.D. of the wire coil electrode 120 is preferably about the same as the outer diameter of the outer tubular sheath 260, the ring electrodes and connector elements and the insulator bands so that the lead 110 has a common outer diameter through its length. The length L is preferably in the range of about 10 mm to about 38 mm and the O.D. is preferably in the range of about 0.5 mm mm to about 2 mm.

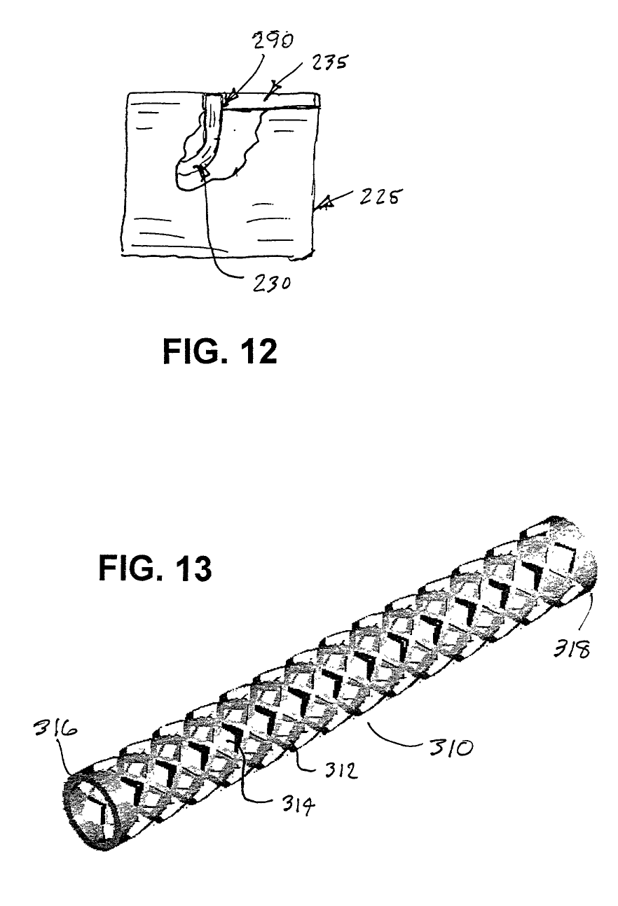

The assembly of the electrode connector 225 and the wire coil 210 is inserted over a portion of the inner sheath 265 to form the exposed wire coil electrode 120 having a coil electrode length, a coil electrode outer diameter O.D., and a coil electrode inner diameter I.D. The electrode connector 225 is in turn connected to the distal end of lead conductor 220 extending proximally through the lead body 115 to connector element 195 in the proximal connector array segment 135 as shown in FIG. 12. An opening. e.g., a slot, 235 is provided in the tubular side wall of the electrode connector 225 that receives the distal end of the lead conductor 230 as shown in FIG. 12. During assembly, the distal end of lead conductor 230 is drawn through the inner sheath 265 and into the slot 235. The distal end of lead conductor 230 is welded at weld 290 into the slot 235. The electrical connections of the distal ends of lead conductors 240, 245 and 255 with ring electrodes 140, 145 and 155 are made in the same manner. The electrical connections of the proximal ends of the lead conductors 240, 220, 245 and 255 with the connector elements 190, 195, 200 and 205 can be made in the same manner.

Thus, the lead 110 is formed having a very small O.D. with at least one elongated distal coil electrode that is highly flexible and capable of conforming to the curvature of he foramen and the sacral nerve extending anteriorly and or posteriorly therefrom. The distal electrode array segment 160 can be percutaneously introduced through the foramen through a percutaneous lead introducer tool set. It will also be understood that the distal tip 130 can be eliminated to provide a through lumen for guide wire introduction of the lead 110 over a guide wire previously extended through the foramen.

It should be noted that the wire coil 210 can be close-wound as shown in the figures or space-wound with a spacing between the turns. Other flexible tubular electrode structures can also be substituted for the wire coil. For example, an elongated, tubular, stent-like tube 310 of the type depicted in FIG. 13 can be substituted for the wire coil 210. The mesh tube 310 can be laser-etched from a thin solid tube of one of the above-mentioned bio-compatible conductive materials. The laser etching removes material to form a lattice 312 framing windows 314 through most of its length between the solid end rings or bands 316 and 318. The connection with the conductor distal end can be made to one of the solid end rings or bands 316 and 318 in the manner described above with reference to FIG. 12, or it may be made in other ways.

In use, the elongated distal lead segment bearing the elongated wire coil electrode 120 or mesh electrode 310 and at least one ring electrode proximal and distal to it, like ring electrodes 140 and 145, is inserted through the foramen to attempt to locate the flexible elongated wire coil electrode 120 or mesh electrode 310 adjacent to or in contact with the sacral nerve. Test stimuli are applied to each electrode in return and a physiologic response of the patient is noted. The response to the test stimuli delivered through the elongated wire coil electrode 120 or mesh electrode 310 should be maximal when it is located relative to the sacral nerve. In this location, the responses to test stimuli delivered through the distal and proximal electrodes 140 and 145 should be noticeably lesser in intensity and about equal.

The true spirit and scope of the inventions of this specification are best defined by the appended claims, to be interpreted in light of the foregoing specification. Other apparatus that incorporates modifications or changes to that which has been described herein are equally included within the scope of the following claims and equivalents thereof. Therefore, to particularly point out and distinctly claim the subject matter regarded as the invention, the following claims conclude this specification.

We claim:

1. An implantable medical lead for non-direct contact electrical stimulation of the sacral nerves comprising:
a lead body extending between lead proximal and distal ends, the lead body comprising a proximal connector element, a distal wire coil electrode, and a lead conductor extending between the connector element and the wire coil electrode, the wire coil electrode further comprising a ring-shaped electrode connector element extending around the lead body and electrically connected to the lead conductor, an elongated, flexible, wire coil extending between first and second coil ends and over a segment of the lead body and electrically insulated from the lead conductor, the electrode connector element coupled to the electrode connector in an annular connection zone providing mechanical and electrical connection,
whereby the wire coil electrode is capable of being inserted through a foramen of the sacrum into operative relation with a sacral nerve to provide stimulation to the sacral nerve without necessarily being in direct contact with the sacral nerve.

2. The implantable medical lead of claim 1, wherein the wire coil and connector element have a common outer diameter and inner diameter and are axially aligned and coupled together in the annular connection zone

3. The implantable medical lead of claim 1, wherein the wire coil and connector element have a common outer diameter and inner diameter and are axially aligned and butt-welded together in the annular connection zone

4. The implantable medical lead of claim 1, wherein the wire coil and connector element have a common outer diameter and inner diameter and are axially aligned and adhered together in the annular connection zone

5. The implantable medical lead of claim 1, wherein the ring-shaped electrode connector is formed of a solid tube side wall with an opening through the side wall that the distal end of the lead conductor is extended into and attached to the side wall.

6. The implantable medical lead of claim 1, wherein the length of the wire coil electrode is preferably in the range of about 10.0 mm to about 38.0 mm and the lead body outer diameter is preferably in the range of about 0.5 mm to about 2 mm.

7. An implantable medical lead for non-direct contact electrical stimulation of the sacral nerves comprising:
a lead body extending between lead proximal and distal ends, the lead body comprising a first proximal connector element, a distal wire coil electrode, and a first lead conductor extending between the first proximal connector element and the wire coil electrode, the wire coil electrode further comprising a ring-shaped electrode connector element extending around the lead body and electrically connected to the lead conductor, an elongated, flexible, wire coil extending between first and second coil ends and over a segment of the lead body and electrically insulated from the lead conductor, the electrode connector element coupled to the electrode connector in an annular connection zone providing mechanical and electrical connection,
the lead body further comprising a second proximal connector element, a distal ring-shaped electrode spaced apart from the distal wire coil electrode, and a second lead conductor extending between the second proximal connector element and the distal ring-shaped electrode,
whereby the wire coil electrode is capable of being inserted through a foramen of the sacrum into operative relation with a sacral nerve to provide stimulation to the sacral nerve without necessarily being in direct contact with the sacral nerve.

8. The implantable medical lead of claim 7, wherein the wire coil and connector element have a common outer diameter and inner diameter and are axially aligned and coupled together in the annular connection zone

9. The implantable medical lead of claim 7, wherein the wire coil and connector element have a common outer diameter and inner diameter and are axially aligned and butt-welded together in the annular connection zone

10. The implantable medical lead of claim 7, wherein the wire coil and connector element have a common outer diameter and inner diameter and are axially aligned and adhered together in the annular connection zone

11. The implantable medical lead of claim 7, wherein the ring-shaped electrode connector is formed of a solid tube side wall with an opening through the side wall that the distal end of the lead conductor is extended into and attached to the side wall.

12. The implantable medical lead of claim 7, wherein the distal ring-shaped electrode is positioned distal to the wire coil electrode.

13. The implantable medical lead of claim 7, wherein the distal ring-shaped electrode is positioned proximal to the wire coil electrode.

14. The implantable medical lead of claim 7, wherein:
the distal ring-shaped electrode is positioned distal to the wire coil electrode,
the lead body further comprises a third proximal connector element, a further ring-shaped electrode positioned proximal to the wire coil electrode, and a third lead conductor extends between the third proximal connector element and the further ring-shaped electrode.

15. The implantable medical lead of claim 7, wherein the length of the wire coil electrode is preferably in the range of about 10.0 mm to about 38.0 mm and the lead body outer diameter is preferably in the range of about 0.5 mm to about 2.0 mm.

16. An implantable medical lead for non-direct contact electrical stimulation of the sacral nerves comprising:
a lead body extending between lead proximal and distal ends, the lead body comprising a proximal connector element, an elongated distal mesh electrode, and a lead conductor extending between the connector element and the distal electrode, the distal mesh electrode further comprising an elongated tube surrounding the lead body and electrically connected to the lead conductor having a side wall formed of a lattice framing windows extending through the side wall and imparting flexibility to the elongated distal mesh electrode,
whereby the mesh electrode is capable of being inserted through a foramen of the sacrum into operative relation with a sacral nerve to provide stimulation to the sacral nerve without necessarily being in direct contact with the sacral nerve.

17. The implantable medical lead of claim 16, wherein the lead body further comprises a second proximal connector element, a distal ring-shaped electrode spaced from the mesh electrode, and a second lead conductor extending between the second proximal connector element and the distal ring-shaped electrode.

18. The implantable medical lead of claim 17, wherein the distal ring-shaped electrode is positioned distal to the mesh electrode.

19. The implantable medical lead of claim 17, wherein the distal ring-shaped electrode is positioned proximal to the mesh electrode.

20. The implantable medical lead of claim 17, wherein:
the distal ring-shaped electrode is positioned distal to the mesh electrode.
the lead body further comprises a third proximal connector element, a further ring-shaped electrode positioned proximal to the mesh electrode, and a third lead conductor extends between the third proximal connector element and the further ring-shaped electrode.
